(19) United States
(12) Patent Application Publication
(10) Pub. No.: US 2009/0304830 A1
(43) Pub. Date: Dec. 10, 2009

(54) Composition Comprising Bamboo Extract for Androgen Agonist

(75) Inventors: Tae-Hyung Jo, Gyeonggi-do (KR); Sung-Sick Woo, Seoul (KR); Ji-Min Cha, Seoul (KR); Dong-Seon Kim, Daejeon (KR); Sun-Young Sung, Gyeonggi-do (KR); Seon-Gil Do, Chungcheongbuk-do (KR); Young-Chul Lee, Daejeon (KR); Jeong-Bum Nam, Chungcheongbuk-do (KR)

Correspondence Address: SWANSON & BRATSCHUN, L.L.C., 8210 SOUTHPARK TERRACE, LITTLETON, CO, 80120, US

(73) Assignee: Unigen, Inc., Chungcheongnamdo (KR)

(21) Appl. No.: 12/544,640

(22) Filed: Aug. 20, 2009

Related U.S. Application Data
(62) Division of application No. 11/629,087, filed on Dec. 11, 2006.

(30) Foreign Application Priority Data
Jun. 11, 2004 (KR) 10-2004-0043020

(51) Int. Cl. A61K 36/899; A61P 3/00; A61P 19/08; A61P 21/00; A61P 25/00; A61P 15/00

(52) U.S. Cl. 424/750

(57) ABSTRACT

The present invention relates to a use of bamboo or bamboo extract to prevent or treat symptoms related to decrease of androgen; a composition for androgen agonist comprising bamboo or bamboo extract; a method for preventing or treating symptoms related to decrease of androgen by administering composition for androgen agonist; and a method of preparing composition for androgen agonist. The present composition obtained from natural material can be used as Phyto-androgen for the preventing and treating symptoms of male climacteric without dangerousness according to hormone replacement therapy.

### RELATED APPLICATIONS

This application divisional of U.S. application Ser. No. 11/629,087, filed Dec. 11, 2006, which is a 35 U.S.C. § 371 national phase application of International application serial no. PCT/KR2005/001765, filed on Jun. 10, 2005 (WO 2005/120537), both of which are entitled “Composition Comprising Bamboo Extract for Androgen Agonist”. International application serial no. PCT/KR2005/001765 claims the benefit of Korean Patent Application Serial No. KR 10-2004-0043020, filed on Jun. 11, 2004. Each of these applications is specifically incorporated herein by reference in its entirety.

### TECHNICAL FIELD

The present invention relates to a use of bamboo or bamboo extract to prevent or treat symptoms related to decrease of androgen; a composition for androgen agonist comprising bamboo or bamboo extract; a method for preventing or treating symptoms related to decrease of androgen by administering a therapeutically effective amount of bamboo or bamboo extract to mammal; and a method of preparing a composition for androgen agonist by extracting bamboo with polar solvent or non-polar solvent.

### BACKGROUND ART

Androgen known as male hormone is one of steroid hormones, and is known to conduct the physiological function and physiological control function through medium of androgen receptor (AR) which is distributed over various tissues such as reproductive system of seminal glands, testis, etc., central nervous system, cardiovascular system, immune system, digestive system, kidney, lung, etc. [Heinlein C A and Chang C, Endocrine Reviews, 2002, 23(2), 175-200]. In terms of function, androgen is produced in seminal glands, arrives at aimed cells through blood vessel, enters the aimed cells by simple diffusion, and affects the transcription activity of the aimed gene through androgen receptor which is the transcription factor in the nucleus [Heinlein C A and Chang C, Endocrine Reviews, 2004, 25(2), 276-308]. It is known that androgen receptor is one of steroid hormone receptors such as glucocorticoid receptor, progesterone receptor, estrogen receptor, and mineralcorticoid receptor, and testosterone, cortisol, progesterone, estradiol, aldosterone, etc., and works as counter ligand thereof [Beato M and Klug J, Human Reproduction Update 2000, 6, 225-236]. In the male as well as the female, the climacteric occurs from before and after 50, and the frequency of occurrence is increased in years. After 60, in 30% of the male, the climacteric occurs (Schneider HPG, Annals of New York Academy Science, 2003, 997, 292-306). However, male climacteric symptoms are exhibited very slowly, and thus many men may not feel any. Also, many men feel climacteric symptoms, but think them due to stress or natural change by aging. One cause of male climacteric is decrease of male hormone due to decrepitude of brain and testis in addition to other causes (Schneider HPG, Annals of New York Academy Science, 2003, 997, 292-306). Main symptoms of male climacteric are fatigue, decline of memory, melancholia, decline of muscular strength, increase of body fat, and weakening of bone. Also, the sexual dysfunction, impotence, and decline of sexual desire, etc. are usually accompanied. Moreover, it is known that lack of androgen results in many symptoms such as decline of sexual desire, impotence, decline of muscle, decline of physical strength, increase of body fat, change of hair, decline of bone density, etc.

If androgenic drug is administered to patients showing the above symptoms, it is shown to be effective for reducing of body fat, increase of muscle, increase of bone density, increase of hand-grip strength, improvement of mood, reduction of melancholia, increase of sexual desire, improvement of the quality of life in AIDS patients, etc. (Bhasin S and Bremner W, Journal of Clinical Endocrinology and Metabolism, 1997, 82, 3-8). Besides, it was reported that androgen inhibits the phosphorylation of tau protein which is the cause of dementia, and thus is applicable for preventing dementia (Papasozomenos Sch, Shanavas A, Proceedings of National Academy of Science 2002, 99, 1140-1145).

Bamboo is a species of Gramineae, and 280 kinds of bamboo are known worldwide, and 70 kinds of bamboo grow in nature or are cultivated in South Korea. The 11 representative kinds of bamboo consist of P. nigra var. henonis; P. bambusoides; P. pubescence; P. nigra; P. nigra for. punctata; S. borealis var. gracilis; S. coreana Nakai; S. borealis var. chiisanensis; S. borealis; S. borealis Makino; and P. japonica etc. Among them, the mainly cultivated kinds are P. bambusoides; P. nigra var. henonis and P. pubescence.

Since the ancient, the bamboo's bark, branch, leaf, sprout, endoderm as B. Caulis in Taeniis, etc. are used as a Chinese medicine material. In particular, Bambusae Caulis in Taeniis is middle layer of Phyllostachys nigra var. henonis or Phyllostachys bambusoides Sieb. et Zucc. whose outer bark is removed from, and is known to have a pharmacological effect for vomiting, removal of phlegm, haemostasis, and comforting embryo. Tabasheer extracted by heating bamboo is reported to be effective for treating palsy and hypertension in Donguibogam, Botanical List, and Encyclopedia of Chinese Medicine, and is reported to be effective for the treatment of hypertension, atherosclerosis, cardiovascular disease, etc. or the prevention of cancer and aging. According to Donguibogam, Botanical List, and Encyclopedia of Chinese Medicine, bamboo is effective for the treatment of palsy and hypertension, and particularly used for thirst in pneumonia, bronchitis, etc. to alleviate fever, discharge phlegm, and refresh. Recently, it is reported that bamboo is effective for the treatment of hypertension, atherosclerosis, and cardiovascular disease, and is introduced to be good for anticancer and prevention of aging. These functions of bamboo are regarded as closely related to the antioxidant effect. Also, phytochemicals like organic acid, dietary fiber, tannin, and benzofurane, existing in bamboo extract are expected to contribute to the prevention of circulatory system disorders through antioxidant function, thorombolysis, lipid reduction function, etc.

At present, the kinds of bamboo naturally growing in South Korea may be divided into Phyllostachys, Sasan and Pseudosasa. In case of Phyllostachys, the leaf sheath is fallen early, the number of stamen is 3, the height is 10-30 cm, the diameter is 3-20 cm, the stem is big, and two buds come out from each joint thereof. In the world, there are 40 kinds of Phyllostachys, which are mainly in China and India, and some of which are in Japan, Europe, and North Africa. In South Korea, 6 kinds of bamboo habitant are known: Phyllostachys pubescence, P. nigra, P. nigra var. henonis, P. nigra for. punctata, P. comprossa, and P. bambusoides.

1) Phyllostachys nigra var. henonis is perennial evergreen shrub and a mutated species of Phyllostachys nigra. The subterranean stem of Phyllostachys nigra var. henonis grows sideward from joints, and its height reaches up to 10 m. The bamboo sprout comes out in April and May, and edible, and its color is brown.

2) The stem color of P. nigra is green in its first year, but becomes black from the second year to be completely black. The height of P. nigra is 3-20 m, the diameter is 2-5 cm, and P. nigra grows forthright. The flower of P. nigra blooms in June and July, and is spike and shaped like an oval having the length of 2.5-3 cm, and the color of flower is purplish green. With a period of about 60 years, P. nigra blooms, bears fruits, and dies.

3) The joint of P. bambusoides has two rings. P. bambusoides grows up to the height of 20 m and the diameter of 5-10 cm. The leaves of P. bambusoides are 5 to 8, and the length of leaf is 10-20 cm. There is fluff at the joint of leaf and stem. The sprout of P. bambusoides is eaten early summer. B. caulis in Taeniis is a thin shell like a piece of paper existing in the inner stem of P. bambusoides, and is used for tooth heat and hematemesis.

4) P. pubescence is called as “juksundae” since juksun (bamboo sprout) coming out in May is favorable to eat, or as “maengjongjuk” which is originated from “Maengjong” who devoted to his parents by serving bamboo sprout in snowy winter. P. pubescence appears to have only one ring on its joint. The fluff at a joint of leaf and stem is fallen, little left. P. pubescence is mainly planted in the southern area.

5) P. nigra for. punctata is a kind of P. nigra. The stem height of P. nigra for. punctata is about 10 m. The stem color is varied depending on environment, but the stem generally has black spot on the yellow base. The flower of P. nigra for. punctata blooms in June and July and is panicle, and many small flower ears thereof are compactly hung thereto.

6) P. comprossa is characterized in that the first joint of branch is flatly pressed, and its seed leaf has fine hairs. The flower of P. comprossa is panicle, and several small flower ears thereof are hung thereto.

The leaf sheath of Sasa has soft or hard, long hairs. The bag of flower ears of Sasa is long, the number of stamen is 3 or 6, the height is 0.3-5 m, the diameter is 2-15 m, and the size is small. 200 kinds of Sasa are distributed over East Asia such as Korea, China, Japan, etc., and some examples thereof are Sasa coreana Nakai, S. coreana, S. kurilensis, S. quelpaertensi), S. borealis, S. borealis var. chiisanensis, S. borealis var. gracilis, etc.

1) Sasa coreana Nakai is an endemic species of Korea, distributed over Myeongcheon, Hamkyeongbuk-do, and grows at the foot of mountain in a group. The height of Sasa coreana Nakai is 30-80 cm, and the diameter is 3-8 mm. The root stock is short, the branch is divided, and the gap of joint is short. The branches mainly come out of at the height of 5-20 cm, and the stem and branches are grooved. The 5 to 8 leaves of Sasa coreana Nakai hang at the end of branch, each shaped like a long oval or egg shape. The length of leaf is 3-12 cm, and the width is 6-22 mm. The front side of leaf does not have any fluff, but the back side of leaf has much fluff, serrate leaf, and 5-6 types of leaf venation. Most of the leave sheaths do not have fluff. The leaf of Sasa coreana Nakai is similar to, but smaller than, that of S. kurilensis, and the branch of Sasa coreana Nakai is denser than that of S. kurilensis. It is known that the leaf of Sasa coreana Nakai is used for hemostatic, expectorant, and diuretic, particularly nephritis, in the oriental medicine.

2) Sasa borealis Makino is perennial evergreen shrub, and grows up to the height of 1-2 m. The bract surrounds the stem for 2 or 3 years, and has fluff. The leaves come out of at the end of branch by twos to threes, and the shape of leaf is long oval and lanceolate. The length of leaf is 10-25 cm, and the leaf is acuminate or long like a tail. The basipetal and sheath of the back side of leaf have fluff. Serrate like a prickle exists at the edge of leaf. The flower blooms in April every five years, and then dies after blooming. The fruit ripens in May and June.

Pseudosasa grows at the foot of mountain of the southern area or plain in group, and is raised for ornament. One branch comes out of each joint every two years. A new sprout thereof comes out wrapped in a shell having tough fluff at the end of subterranean stem. The bark is longer than the gap between joints. The length of leaf is about 30 cm. The flower blooms from late spring to summer. Pseudosasa consists of two kinds: P. japonica and P. japonica var. purpurascens.

1) Pseudosasa japonica mainly grows in the central and southern area of Korea. The height is 2-4 m, the diameter is 5-15 mm, and 5-6 branches come out of the upper middle part. The leaf is lanceolate and has no fluff. The length of leaf is 10-30 cm, and the width is 1-4 cm. The flower is coniform, and 5 to 10 of small petals thereof come out. The bamboo sprout comes out in May.

2) Pseudosasa japonica var. purpurascens is a mutated species of P. japonica, meaning that the leafstalk and leaf is purple colored, and grows in Cheju Island.

### DISCLOSURE OF THE INVENTION

The present inventors searched natural products acting as a transcription activating factor through androgen receptor by observing reporter gene expression through ARE (Androgen Receptor Element) using ARE4-Luc reporter plasmid, to develop androgen agents from natural products, distinguishably from the development strategy in the developed countries. They experimented to develop a material similar to androgen whose activity is proven by using natural product library, and then found out that the bamboo extract shows androgen activity, to complete the present invention.

Thus, an object of the present invention is to provide a new composition for androgen agonist comprising bamboo or bamboo extract.

Another object of the present invention is to provide a use of bamboo or bamboo extract to prevent or treat symptoms related to decrease of androgen.

Another object of the present invention is to provide a method of prevention or treatment of symptoms related to decrease of androgen by administering a therapeutically effective amount of bamboo or bamboo extract to mammal.

Another object of the present invention is to provide a method for preparing a composition for androgen agonist by extracting bamboo with polar solvent or non-polar solvent.

### BRIEF DESCRIPTION OF THE DRAWINGS

FIG. 1 is a graph showing the androgen activity of bamboo alcoholized extract.

FIG. 2 is a graph showing the androgen activity of B. caulis in Taeniis extract and solvent fraction thereof.

FIG. 3 is a graph showing the androgen activity of S. coreana Nakai extract and solvent fraction thereof.

FIG. 4 is a graph showing the androgen activity of P. nigra var. henonis extract and solvent fraction thereof.

FIG. 5 is a graph showing the androgen activity of P. japonica extract and solvent fraction thereof.

FIG. 6 is a graph showing the androgen activity of bamboo hydrothermal extract.

### BEST MODE FOR CARRYING OUT THE INVENTION

According to the above objects, the present invention provides a composition for androgen agonist comprising bamboo or bamboo extract as effective ingredient.

The present invention also provides a use of bamboo or bamboo extract to prevent or treat symptoms related to decrease of androgen.

The present invention also provides a method of prevention or treatment of symptoms related to decrease of androgen by administering a therapeutically effective amount of bamboo or bamboo extract to mammal.

The present invention also provides a method for preparing a composition for androgen agonist by extracting bamboo with polar solvent or non-polar solvent.

In the present composition, it is preferable to select bamboo from the group comprising Phyllostachys, Sasa, or Pseudosasa; the Phyllostachys bamboo is preferably selected from Phyllostachys nigra var. henonis, P. nigra, P. bambusoides, P. pubescence, P. nigra for. Punctata, or P. comprossa; the Sasa bamboo is preferably selected from Sasa coreana Nakai, S. coreana, S. kurilensis, S. quelpaertensis, S. borealis, S. borealis var. chiisanensis, or S. borealis var. gracilis; the Pseudosasa bamboo is preferably selected from Pseudosasa japonica, or Pseudosasa japonica var. purpurascens, and bamboo can be used by root, stem, leaf, or herb.

In the present composition, bamboo can be used by herb, branch, shell, leaf, sprout, root, endodermis, etc., preferably used in the form of powder or extract.

The bamboo extract can be used by extracting bamboo with water, organic solvent, or mixing solvent thereof.

All solvents can be used as the above organic solvent, preferably polar solvent such as water, C₁₋₄ alcohol, etc., or non-polar solvent such as n-hexane, dichloromethane, etc.

The above non-polar solvent extract of bamboo comprises extract extracted with non-polar solvent selected from n-hexane, dichloromethane, chloroform, or ethylacetate, preferably n-hexane, dichloromethane, and ethylacetate.

The above polar solvent extract of bamboo comprises extract extracted with polar solvent selected from acetone, water, or C₁₋₄ alcohol such as methanol, ethanol, propanol, butanol, etc.

The present bamboo extract also may be water fraction or n-hexane fraction obtained by suspending the above C₁₋₄ alcohol extract with water and adding n-hexane thereto; dichloromethane fraction obtained by adding dichloromethane to the above water fraction; ethylacetate fraction obtained by adding ethylacetate to water fraction remaining after separation of the above dichloromethane fraction; n-butanol fraction obtained by adding n-butanol to water fraction remaining after separation of the above ethylacetate fraction; or extract obtained by column chromatography of the above extracts and fractions.

A process for extracting bamboo of the present invention is specifically described in a following example.

B. caulis in Taeniis is sliced to small pieces, and then water, methanol, or ethanol in the amount of 5 or 25 folds of dry weight of the pieces are added thereto, and extracted under reflux condenser to obtain water extract, methanol extract, or ethanol extract of B. caulis in Taeniis. To the above methanol or ethanol extract is added distilled water, and the mixture is suspended and fractioned by adding N-hexane thereto to obtain water soluble fraction and n-hexane soluble fraction. Also, to the water fraction remaining after separation of the n-hexane soluble fraction is added dichloromethane to obtain dichloromethane fraction. Again, to the water fraction remaining after separation of the dichloromethane fraction is added ethylacetate to obtain ethylacetate fraction. Also, to the water fraction remaining after separation of the ethylacetate fraction is added n-butanol to obtain n-butanol fraction. Then, the above extracts and fractions are separated by chromatography to obtain purified extract.

The above extraction may be carried out by conventional methods such as hot water extraction or sonication. Lyophilized product of the extract can be used for the present composition.

The present composition can be used as androgen agonist and phyto-androgen obtained from natural materials. Thus, the present composition can be used for the treatment and prevention of male climacteric, specifically reduction of body fat, increase of muscle, increase of bone density, increase of hand-grip strength, improvement of mood, reduction of melancholia, increase of sexual desire, or prevention or treatment of dementia.

The composition of the present invention can be prepared according to conventional methods in the pharmaceutical field into conventional pharmaceutical preparations, for example, solution such as drinks, syrup and capsule, by mixing with pharmaceutically acceptable carrier, excipient, etc.; and administered orally or parenterally. Preferably, the composition of the present invention may be orally administered in drink before and/or after the meal for quick effect.

Capsule and solution comprising the composition of the present invention may be used as medicine or health care products. Here, “health care products” mean food products prepared and processed in the form of tablet, capsule, powder, granule, solution, pill, etc., by using material or ingredients having useful function to the human body.

The composition of the present invention is appropriately administered according to the extent of absorption of active ingredients into the body; excretion rate; age, weight, sex, and condition of patient; severity of treated disease, etc. However, generally, in solution, it is preferable to administer the present composition 1˜3 times a day, 0.01˜500 mg/kg, preferably 0.1˜200 mg/kg each to adult. In other preparations, an appropriate amount based on the above dose for solution can be administered orally.

Hereinafter, the present invention will be described in more detail with reference to the following examples, but the scope of the present invention should not be construed to be limited thereby in any manner.

### EXAMPLES

### Example 1

### Preparation of B. caulis in Taeniis Alcoholized Extract

1-1) B. caulis in Taeniis used in the experiment was produced in Korea, and purchased in the Kyung-Dong market. The purchased B. caulis in Taeniis was washed by clean water, and air-dried to be used as sample for extraction. To 1 kg of B. caulis in Taeniis made into small fragments after the drying was added 15 L of 70% ethanol in the amount of 15 folds of the dry weight of B. caulis in Taeniis, which was continuously repeatedly extracted three times at a constant interval (every 12 h) at 80° C., and then filtered under reduced pressure with filter paper (Watman Co., USA). The filtrate was collected and concentrated under reduced pressure by vacuum rotation at 60° C., and thus extracted residue was dried by lyophilizer to obtain 72 g of B. caulis in Taeniis crude extract, which was kept in a freezer of −20° C., and used in the experiment.

1-2) Preparation of B. caulis in Taeniis n-Hexane Soluble Fraction

To 50 g of crude extract of B. caulis in Taeniis obtained in the above 1-1) was added 1 L of distillated water, and the mixture was suspended and mixed by adding n-hexane 11, and then repeatedly fractioned three times to obtain 2 L of water soluble fraction and 2 L of n-hexane soluble fraction. And, the n-hexane soluble fraction was filtered, and dried under reduced pressure to obtain 10.2 g of B. caulis in Taeniis dried powder of n-hexane soluble fraction, which was used as sample.

1-3) Preparation of B. caulis in Taeniis Dichloromethane Soluble Fraction

To 2 L of water soluble fraction obtained in the above 1-2) was added 1 L of dichloromethane and mixed, and then the mixture was repeatedly fractioned three times to obtain 2 L of water soluble fraction and 2 L of dichloromethane soluble fraction. Then, the dichloromethane soluble fraction was filtered and dried under reduced pressure to obtain 8.1 g of B. caulis in Taeniis dried powder of dichloromethane soluble fraction, which was used as sample.

1-4) Preparation of B. caulis in Taeniis Ethylacetate Soluble Fraction

To 2 L of water soluble fraction obtained in the above 1-3) was added 1 L of ethylacetate and mixed, and then the mixture was repeatedly fractioned three times to obtain 2 L of water soluble fraction and 2 L of ethylacetate soluble fraction. Then, the ethylacetate soluble fraction was filtered and dried under reduced pressure to obtain 5.5 g of B. caulis in Taeniis dried powder of ethylacetate soluble fraction, which was used as sample.

1-5) Preparation of B. caulis in Taeniis n-Butanol Soluble Fraction

To 2 L of water soluble fraction obtained in the above 1-4) was added 1 L of n-butanol and mixed, and then the mixture was repeatedly fractioned three times to obtain 2 L of water soluble fraction and 2 L of n-butanol soluble fraction. Then, the n-butanol soluble fraction was filtered and dried under reduced pressure to obtain 7.1 g of B. caulis in Taeniis dried powder of n-butanol soluble fraction and 13.5 g of B. caulis in Taeniis dried powder of water soluble fraction.

### Example 2

### Preparation of B. caulis in Taeniis Hydrothermal Extract

2-1) B. caulis in Taeniis used in the experiment was produced in Korea and purchased in the Kyung-Dong market. The purchased B. caulis in Taeniis was washed by clean water, and air-dried to be used as a sample for extraction. To 2 kg of B. caulis in Taeniis made into small fragments after the drying was added 30 L of purified water in the amount of 15 folds of the dry weight of B. caulis in Taeniis, which was continuously repeatedly extracted two times at a constant interval (every 10 h) at 80° C., and then filtered under reduced pressure with filter paper (Watman Co., USA). The filtrate was collected and concentrated under reduced pressure by vacuum rotation at 60° C., and thus extracted residue was dried by lyophilizer to obtain 87 g of B. caulis in Taeniis crude extract, which was kept in a freezer of −20° C., and used in the experiment.

### Example 3

### Preparation of S. coreana Nakai Alcoholized Extract

3-1) S. coreana Nakai used in the experiment was purchased in Daebat Goeul, Seojeong-ri, Gonyang-myeon, Sacheon-si, Gyeongsangnam-do, Korea. The purchased S. coreana Nakai was washed by clean water, and air-dried to be used as a sample for extraction. To 2 kg of S. coreana Nakai made into small fragments after the drying was added 30 L of 70% ethanol in the amount of 15 folds of the dry weight of S. coreana Nakai, which was continuously repeatedly extracted three times at a constant interval (every 12 h) at 80° C., and then filtered under reduced pressure with filter paper (Watman Co., USA). The filtrate was collected and concentrated under reduced pressure by vacuum rotation at 60° C., and thus extracted residue was dried by lyophilizer to obtain 71.6 g of S. coreana Nakai crude extract, which was kept in a freezer of −20° C., and used in the experiment.

3-2) Preparation of S. coreana Nakai Solvent Fraction

Each solvent fraction was prepared by using 50 g of crude extract obtained in the above 3-1) with same methods of the above 1-2) to 1-5) of Example 1, to obtain solvent fraction as following Table 1.

TABLE 1

| | | Extract |
|---|---|---|
| | Extract name | yield |
| 3-2) | N-hexane soluble fraction of S. coreana Nakai | 2.5 g |
| 3-3) | Dichloromethane soluble fraction of S. coreana Nakai | 1.2 g |
| 3-4) | Ethylacetate soluble fraction of S. coreana Nakai | 1.4 g |
| 3-5) | N-butanol soluble fraction of S. coreana Nakai | 2.4 g |
| 3-6) | Water soluble fraction of S. coreana Nakai | 42.5 g |

### Example 4

### Preparation of S. coreana Nakai Hydrothermal Extract

4-1) S. coreana Nakai used in the experiment was purchased in Daebat Goeul, Seojeong-ri, Gonyang-myeon, Sacheon-si, Gyeongsangnam-do, Korea. The purchased S. coreana Nakai was washed by clean water, and air-dried to be used as a sample for extraction. To 2 kg of S. coreana Nakai made into small fragments after the drying was added 30 L of purified water in the amount of 15 folds of the dry weight of S. coreana Nakai, which was continuously repeatedly extracted two times at a constant interval (every 10 h) at 80° C., and then filtered under reduced pressure with filter paper (Watman Co., USA). The filtrate was collected and concentrated under reduced pressure by vacuum rotation at 60° C., and thus extracted residue was dried by lyophilizer to obtain 89.6 g of S. coreana Nakai crude extract, which was kept in a freezer of −20° C., and used in the experiment.

### Example 5

### Preparation of S. borealis Alcoholized Extract

5-1) S. borealis used in the experiment was purchased in Daebat Goeul, Seojeong-ri, Gonyang-myeon, Sacheon-si, Gyeongsangnam-do, Korea. The purchased S. borealis was washed by clean water, and air-dried to be used as a sample for extraction. To 1 kg of S. borealis made into small fragments after the drying was added 15 L of 70% ethanol in the amount of 15 folds of the dry weight of S. borealis, which was continuously repeated extracted three times at a constant interval (every 12 h) at 80° C., and then filtered under reduced pressure with filter paper (Watman Co. USA). The filtrate was collected and concentrated under reduced pressure by vacuum rotation at 60° C., and thus extracted residue was dried by lyophilizer to obtain 57 g of S. borealis crude extract, which was kept in a freezer of −20° C., and used in the experiment.

5-2) Preparation of S. borealis Solvent Fraction

Each solvent fraction was prepared by using 50 g of crude extract obtained in the above 5-1) with same methods of the above 1-2) to 1-5) of Example 1, to obtain solvent fraction as following Table 2.

TABLE 2

| | | Extract |
|---|---|---|
| | Extract name | yield |
| 5-2) | N-hexane soluble fraction of S. borealis | 9.5 g |
| 5-3) | Dichloromethane soluble fraction of S. borealis | 4.1 g |
| 5-4) | Ethylacetate soluble fraction of S. borealis | 4.8 g |
| 5-5) | N-butanol soluble fraction of S. borealis | 27.9 g |
| 5-6) | Water soluble fraction of S. borealis | 27.9 g |

### Example 6

### Preparation of S. borealis Hydrothermal Extract

6-1) S. borealis used in the experiment was purchased in Daebat Goeul, Seojeong-ri, Gonyang-myeon, Sacheon-si, Gyeongsangnam-do, Korea. The purchased S. borealis was washed by clean water, and air-dried to be used as a sample for extraction. To 1 kg of S. borealis made into small fragments after the drying was added 15 L of purified water in the amount of 15 folds of the dry weight of S. borealis, which was continuously repeatedly extracted two times at a constant interval (every 10 h) at 80° C., and then filtered under reduced pressure with filter paper (Watman Co. USA). The filtrate was collected and concentrated under reduced pressure by vacuum rotation at 60° C., and thus extracted residue was dried by lyophilizer to obtain 61.4 g of S. borealis crude extract, which was kept in a freezer of −20, and used in the experiment.

### Example 7

### Preparation of P. nigra var. henonis Alcoholized Extract

7-1) P. nigra var. henonis used in the experiment was purchased in Daebat Goeul, Seojeong-ri, Gonyang-myeon, Sacheon-si, Gyeongsangnam-do, Korea. The purchased P. nigra var. henonis was washed by clean water, and air-dried to be used as a sample for extraction. To 1 kg of P. nigra var. henonis made into small fragments after the drying was added 15 L of 70% ethanol in the amount of 15 folds of the dry weight of P. nigra var. henonis, which was continuously repeatedly extracted three times at a constant interval (every 12 h) at 80° C., and then filtered under reduced pressure with filter paper (Watman Co. USA). The filtrate was collected and concentrated under reduced pressure by vacuum rotation at 60° C., and thus extracted residue was dried by lyophilizer to obtain 52 g of P. nigra var. henonis crude extract, which was kept in a freezer of −20° C., and used in the experiment.

7-2) Preparation of P. nigra var. henonis Solvent Fraction

Each solvent fraction was prepared by using 50 g of crude extract obtained in the above 7-1) with same methods of the above 1-2) to 1-5) of Example 1, to obtain solvent fraction as following Table 3.

TABLE 3

| | | Extract |
|---|---|---|
| | Extract name | yield |
| 7-2) | N-hexane soluble fraction of P. nigra var. henonis | 9.1 g |
| 7-3) | Dichloromethane soluble fraction of P. nigra var. | 4.6 g |
| | henonis | |
| 7-4) | Ethylacetate soluble fraction of P. nigra var. henonis | 4.3 g |
| 7-5) | N-butanol soluble fraction of P. nigra var. henonis | 7.1 g |
| 7-6) | Water soluble fraction of P. nigra var. henonis | 25.1 g |

### Example 8

### Preparation of P. nigra var. henonis Hydrothermal Extract

8-1) P. nigra var. henonis used in the experiment was purchased in Daebat Goeul, Seojeong-ri, Gonyang-myeon, Sacheon-si, Gyeongsangnam-do, Korea. The purchased P. nigra var. henonis was washed by clean water, and air-dried to be used as a sample for extraction. To 1 kg of P. nigra var. henonis made into small fragments after the drying was added 15 L of purified water in the amount of 15 folds of dry weight of P. nigra var. henonis, which was continuously extracted two times at a constant interval (every 10 h) at 80° C., and then filtered under reduced pressure with filter paper (Watman Co. USA). The filtrate was collected and concentrated under reduced pressure by vacuum rotation at 60° C., and thus extracted residue was dried by lyophilizer to obtain 57 g of P. nigra var. henonis crude extract, which was kept in a freezer of −20° C., and used in the experiment.

### Example 9

### Preparation of P. japonica Alcoholized Extract

9-1) P. japonica used in the experiment was purchased in Daebat Goeul, Seojeong-ri, Gonyang-myeon, Sacheon-si, Gyeongsangnam-do, Korea. The purchased P. japonica was washed by clean water, and air-dried to be used as a sample for extraction. To 2 kg of P. japonica made into small fragments after the drying was added 15 L of 70% ethanol in the amount of 15 folds of the dry weight of P. japonica, which was continuously repeatedly extracted three times at a constant interval (every 12 h) at 80° C., and then filtered under reduced pressure with filter paper (Watman Co. USA). The filtrate was collected and concentrated under reduced pressure by vacuum rotation at 60° C., and thus extracted residue was dried by lyophilizer to obtain 73.4 g of P. japonica crude extract, which was kept in a freezer of −20° C., and used in the experiment.

9-2) Preparation of P. japonica Solvent Fraction

Each solvent fraction was prepared by using 50 g of crude extract obtained in the above 9-1) with same methods of the above 1-2) to 1-5) of Example 1, to obtain solvent fraction as following Table 4.

TABLE 4

| | | Extract |
|---|---|---|
| | Extract name | yield |
| 9-2) | N-hexane soluble fraction of P. japonica | 2.7 g |
| 9-3) | Ethylacetate soluble fraction of P. japonica | 3.1 g |
| 9-4) | N-butanol soluble fraction of P. japonica | 2.5 g |
| 9-5) | Water soluble fraction of P. japonica | 41.7 g |

### Example 10

### Preparation of P. japonica Hydrothermal Extract

10-1) P. japonica used in the experiment was purchased in Daebat Goeul, Seojeong-ri, Gonyang-myeon, Sacheon-si, Gyeongsangnam-do, Korea. The purchased P. japonica was washed by clean water, and air-dried to be used as a sample for extraction. To 1 kg of P. japonica made into small fragments after the drying was added 15 L of purified water in the amount of 15 folds of the dry weight of P. japonica, which was continuously repeatedly extracted two times at a constant interval (every 10 h) at 80° C., and then filtered under reduced pressure with filter paper (Watman Co. USA). The filtrate was collected and concentrated under reduced pressure by vacuum rotation at 60° C., and thus extracted residue was dried by lyophilizer to obtain 53.3 g of P. japonica crude extract, which was kept in a freezer of −20° C., and used in the experiment.

### Experimental Example

### Test Method for the Activity Analogous to Phytoandrogen

Luciferase plasmid induced by androgen receptor and androgen was introduced into COS cell (Korean Cell Line Bank) to test whether bamboo extract shows the activity analogous to androgen.

The control group was the cell treated with only medium in plasmid made of ARE. The transcription activity value to all the experimental results represents the value of fold, compared with the control group (AR+ARE).

The reagent related to cell culture such as DMEM and the like was purchased from Gibco, and luciferase assay kit was purchased from Promega for use. All the other reagents were purchased from Sigma. pARE4-Luc plasmid and the other plasmids are provided by Dr. Chang in Rochester University, USA, for the present experiment example.

1) Culture of the Cell Line

COS cell was cultured under the conditions of 5% CO₂ and 37° C. in DMEM containing 10% of fetal bovine serum. The medium was exchanged every 2 or 3 days and changed with new one while treating the reagents.

2) Transient Transfection

COS cell was cultured to 70%-80% in 100 mm tissue culture plate, and after removing the medium therefrom, the cell was washed by PBS. After separating the cell from culture plate by treating trypsin, the cell was put into the medium containing serum to inactivate the action of trypsin.

For electroporation, the cell pellet was made into the concentration of 5×10⁶ cells/ml, and was centrifuged by cold PBS. The cell pellet was suspended in 400 μL of PBS, mixed with prepared hAR and pARE4-Luc plasmid, then put into electroporation cuvette to transfect the plasmid into the cell by electric pulse of 250 F, 350V with Gene Pulser (Bio-Rad), left in CO₂ incubator at 37° C. for 10 min, and diluted in DMEM containing 10% of calf serum, to be seeded into 96 well plate. After 24 hours, the medium was changed with new one containing 10% of charcoal-dectran stripped calf serum, and the activity of luciferase was determined after 24 hours from the drug treatment.

3) The Determination of Luciferase Activity

The cell transfected with the plasmid was washed with PBS, and was destroyed by adding lysis buffer (125 mM Tris pH 7.8, 10 mM CDTA, 10 mM DTT, 50% glycerol, 5% Triton X-100), to obtain the supernatant, and the amount of protein therein was quantified by Bradford assay. The luciferase activity was determined after adding 100 μL assay buffer (20 mM Tricine, 1.07 mM (MgCO₃)₄Mg(OH)₂; 5H2O, 2.67 mM MgSO₄, 0.1 mM EDTA, 33.3 mM DTT, 270 μM Coenzyme A(lithium salt), 470 μM Luciferin, 530 μM ATP) to 20 μL of cell extract. The luciferase activity was determined after adding 100 μL assay buffer (20 mM Tricine, 1.07 mM (MgCO₃)₄ Mg(OH)₂; 5H₂O, 2.67 mM MgSO₄, 0.1 mM EDTA, 33.3 mM DTT, 270 μM Coenzyme A(lithium salt), 470 μM Luciferin, 530 μM ATP) to 20 μL of cell extract, and then the light emission was determined for 20 sec with Luminometer (LUMAT LB 9501/16).

Result

1. Androgen Activity of Bamboo Alcoholized Extract

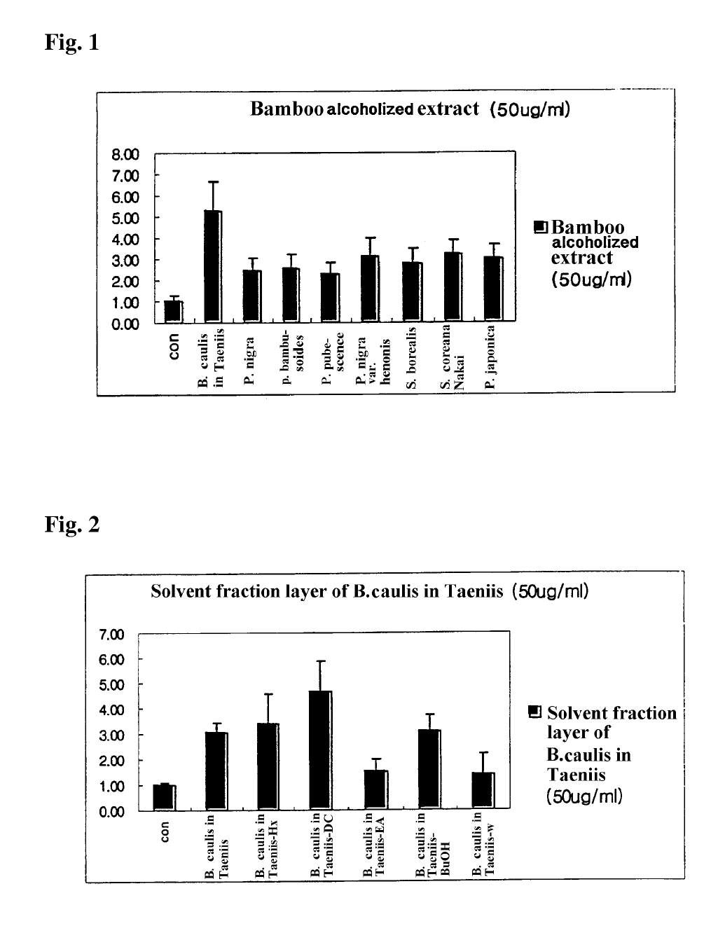

The activity analogous to androgen was determined by using the ethanol crude extract of bamboo prepared in the above Example 1. The results were shown in the FIG. 1. As shown in the FIG. 1, most bamboo extracts show more androgen activity than the control group not treated at all. The activity analogous to androgen of each bamboo extract compared with the control group was as follows: P. nigra (2.45 folds), P. bambusoides (2.57 folds), P. pubescence (2.26 folds), P. nigra var. henonis (3.12 folds), S. borealis (2.76 folds), S. coreana Nakai (3.23 folds), P. japonica (2.98 folds), and B. caulis in Taeniis made with inner shells of P. bambusoides and P. nigra var. henonis (5.29 folds).

2. Androgen Activity of B. caulis in Taeniis Extract and Solvent Fraction Thereof

The activity analogous to androgen of B. caulis in Taeniis extract and solvent fraction thereof processed from Phyllostachys showing the greatest activity among bamboo extracts was determined. The results were shown in the FIG. 2. As shown in the FIG. 2, hexane layer (B. caulis in Taeniis-Hx, 3.37 folds) and dichloromethane layer(B. caulis in Taeniis-DC, 4.67 folds) of B. caulis in Taeniis show the greatest androgen activity. The activity analogous to androgen of extract and each layer of B. caulis in Taeniis compared with the control group was as follows: extract (3.04 folds), ethylacetate layer (B. caulis in Taeniis-EA, 1.53 folds), butanol layer (B. caulis in Taeniis-BuOH, 3.07 folds) and water layer (B. caulis in Taeniis-w, 1.39 folds).

3. Androgen Activity of S. coreana Nakai Extract and Solvent Fraction Thereof.

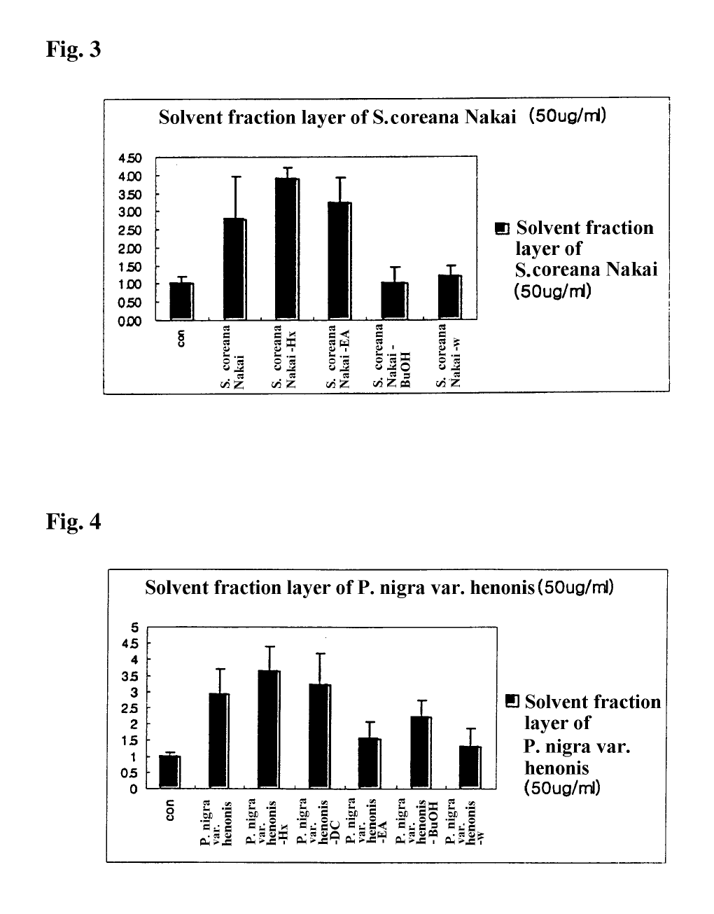

The activity analogous to androgen of S. coreana Nakai extract and solvent fraction thereof prepared from S. coreana Nakai belonging to Sasa was determined. The results were shown in the FIG. 3. As shown in the FIG. 3, activity analogous to androgen of S. coreana Nakai extract was 20.8 folds of that of control. The activity analogous to androgen of each layer of S. coreana Nakai compared with the control group was as follows: Hexane layer (S. coreana Nakai-Hx, 3.92 folds), ethylacetate layer (S. coreana Nakai-EA, 3.22 folds), butanol layer (S. coreana Nakai-BuOH, 1.02 folds) and water layer (1.21 folds).

4. Androgen Activity of P. nigra var. henonis Extract and Solvent Fraction Thereof.

The activity analogous to androgen of P. nigra var. henonis extract and solvent fraction thereof belonging to Phyllostachys was determined. The results were shown in the FIG. 4. As shown in the FIG. 4, activity analogous to androgen of P. nigra var. henonis extract showed 2.92 folds of that of control. The activity analogous to androgen of each layer of P. nigra var. henonis compared with the control group was as follows: hexane layer (P. nigra var. henonis-Hx, 3.63 folds), dichloromethane layer (P. nigra var. henonis-DC, 3.21 folds), ethylacetate layer (P. nigra var. henonis-EA, 1.56 folds), butanol layer (P. nigra var. henonis-BuOH, 2.21 folds) and water layer (P. nigra var. henonis-w, 1.30 folds).

5. Androgen Activity of P. japonica Extract and Solvent Fraction Thereof.

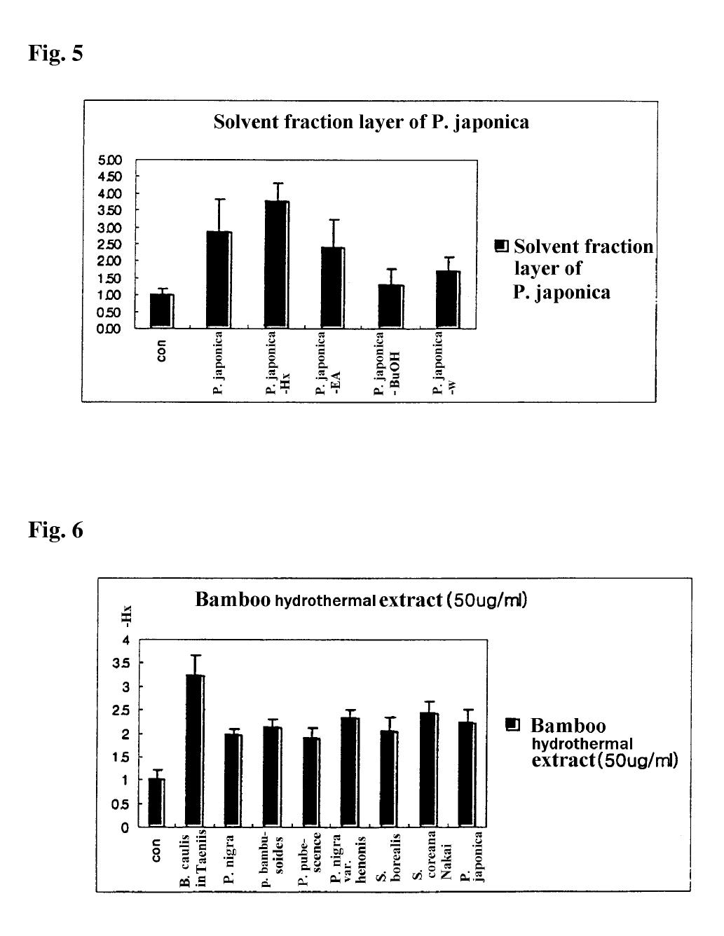

The activity analogous to androgen of P. japonica extract and solvent fraction thereof belonging to Pseudosasa was determined. The results were shown in the FIG. 5. As shown in the FIG. 5, activity analogous to androgen of P. japonica extract showed 2.86 folds of that of control. The activity analogous to androgen of each layer of P. japonica compared with the control group was as follows: hexane layer (P. japonica-Hx, 3.77 folds), ethylacetate layer (P. japonica-EA, 2.39 folds), butanol layer (P. japonica-BuOH, 1.29 folds) and water layer (P. japonica-w, 1.71 folds).

6. Androgen Activity of Bamboo Hydrothermal Extract.

The activity analogous to androgen was determined by using the bamboo hydrothermal extract prepared in the above Example 2, 4, 6, 8 and 10. The results were shown in the FIG. 6. As shown in the FIG. 6, most of bamboo hydrothermal extract showed more androgen activity than the control group not treated at all. The activity analogous to androgen of each bamboo hydrothermal extract compared with the control group was as follows: P. nigra (1.95 folds), P. bambusoides (2.11 folds), P. pubescence (1.89 folds), P. nigra var. henonis (2.32 folds), S. borealis (2.05 folds), S. coreana Nakai (2.42 folds), P. japonica (2.21 folds), and B. caulis in Taeniis made with inner shell of P. bambusoides and P. nigra var. henonis (3.23 folds).

### Formulation Example 1

### Preparation of Solution

| | |
|---|---|
| B. caulis in Taeniis ethanol extract of Example 1 | 20 g |
| Sugar | 10 g |
| Isomerized sugar | 10 g |
| Smell of lemon | proper quantity |
| Total amount after adding purified water | 100 ml |

The above-mentioned ingredients were mixed according to conventional preparation method for solution, and sterilized to give solution.

### Formulation Example 2

### Preparation of Solution

| | |
|---|---|
| S. coreana Nakai ethylacetate fraction of Example 2 | 30 g |
| Sugar | 10 g |
| Isomerized sugar | 10 g |
| Smell of lemon | proper quantity |
| Total amount after adding purified water | 100 ml |

The above-mentioned ingredients were mixed according to conventional preparation method for solution, and sterilized to give solution.

### Formulation Example 3

### Preparation of Capsule

| | |
|---|---|
| P. nigra var. henonis dichloromethane | 500 mg |
| fraction of Example 4 | |
| Lactose | 50 mg |
| Starch | 50 mg |
| Talc | 2 mg |
| Magnesium Stearate | proper quantity |

The above-mentioned ingredients were mixed, and filled in a gelatin capsule according to conventional preparation method for capsule to give capsule.

### Formulation Example 4

### Preparation of Capsule

| | |
|---|---|
| P. japonica ethanol extract of Example 5 | 500 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to conventional preparation method for capsule to give capsule.

### Formulation Example 5

### Preparation of Capsule

| | |
|---|---|
| P. japonica hydrothermal extract of Example 10 | 10 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to conventional preparation method for capsule to give capsule.

### INDUSTRIAL APPLICABILITY

The present composition comprising bamboo extract can be used for androgen agonist because androgen activity of the composition is outstanding. Also, the present composition obtained from natural material can be as a medicine or health care product for the prevention and treatment of male climacteric without dangerousness according to hormone replacement therapy.

1. A method for prevention or treatment of symptoms related to a decrease of androgen comprising administering a therapeutically effective amount of bamboo or bamboo extract to mammal.

2. The method according to claim 1, wherein the extract is extracted with polar solvent such as water, acetone, or C₁₋₄ alcohol, or mixture thereof.

3. The method according to claim 1, wherein the extract is extracted with a non-polar solvent such as n-hexane, dichloromethane, or ethyl acetate.

4. The method according to claim 1, wherein the bamboo is selected from Phyllostachys, Sasa, or Pseudosasa.

5. The method according to claim 4, wherein the Phyllostachys bamboo is selected from Phyllostachys nigra var. henonis, P. nigra, P. bambusoides, P. pubescence, P. nigra for. punctata or P. comprossa; the Sasa bamboo is selected from Sasa coreana Nakai, S. coreana, S. kurilensis, S. quelpaertensis, S. borealis, S. borealis var. chiisanensis or S. borealis var. gracilis; and the Pseudosasa bamboo is selected from Pseudosasa japonica or Pseudosasa japonica var. purpurascens.

6. The method according to claim 1, wherein the extract is selected from the group consisting of a water fraction or a n-hexane fraction obtained by suspending a C₁₋₄ alcohol extract with water, adding n-hexane thereto, and distributing; a dichloromethane fraction obtained by adding dichloromethane to the above water fraction, and distributing; an ethyl acetate fraction obtained by adding ethyl acetate to the water fraction remaining after separation of the above dichloromethane fraction, and distributing; an n-butanol fraction obtained by adding n-butanol to the water fraction remaining after separation of the above ethyl acetate fraction, and distributing; or an extract obtained by column chromatography of the above extracts and fractions.

7. The method according to any of claims 1, wherein the symptom related to decrease of androgen is symptom of male climacteric.

8. The method according to claim 7, wherein the symptom of male climacteric is selected from increase of body fat, decline of muscle, decline of bone density, decline of hand-grip strength, decline of mood, melancholia, decline of sexual desire or dementia.
